(19) United States
(12) Patent Application Publication
(10) Pub. No.: US 2012/0142788 A1
(43) Pub. Date: Jun. 7, 2012

(54) COSMETIC PREPARATION CONTAINING ACYL ARGINATES

(75) Inventors: Manuela Koehler, Hamburg (DE); Antonia Wagner, Hamburg (DE); Kerstin Skubsch, Prisdorf (DE)

(73) Assignee: BEIERSDORF AG, Hamburg (DE)

(21) Appl. No.: 13/381,441

(22) PCT Filed: May 5, 2010
(86) PCT No.: PCT/EP2010/002738

§ 371 Date: Feb. 14, 2012

(30) Foreign Application Priority Data
Jul. 20, 2009 (DE) 102009034115.3

(51) Int. Cl. A61K 8/92; A61Q 19/00; C07C 279/14

(52) U.S. Cl. 514/784; 554/53

(57) ABSTRACT

Cosmetic or dermatological emulsions with a content of one or more acyl arginates of the formula, wherein R can have values from 5 through 17 and preferably represents an unbranched alkyl chain of 11 C atoms (=lauroyl arginate).

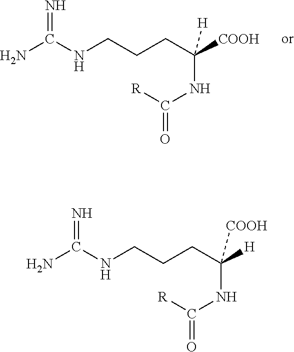

The present invention relates to cosmetic preparations with a content of acyl arginates.

Cosmetics can be used to cover all measures that for esthetic reasons make changes to the skin and hair or are used for personal hygiene. Cosmetics thus means to care for, to improve and to beautify one's physical appearance in order to please others as well as oneself in terms of appearance, touch and scent. Thousands of years ago cosmetics were already being used for this purpose. Color was applied to the lips and face, valuable oils were applied and baths taken in scented water.

While the cleansing effect of a shampoo, the improvement in combability of a conditioner or the curl of the hair due to a permanent wave treatment can be established easily and objectively, other effects and properties of cosmetic properties are virtually impossible to measure or are perceived only very individually. This includes, for example, a certain feeling on the skin (invigorating, soft, silky, smooth, etc.) or the softness and elasticity of the skin after the application of a cosmetic and the thickness and bounce of the hair and the like. Furthermore, consumers' expectations are also subject to subordinate properties of the product. In particular, the scent and the color of the cosmetic as well as its packaging, price, manufacturer and advertising should be mentioned here.

The term “sensorics” refers to the scientific discipline that is concerned with the evaluation of cosmetic preparations based on sensory impressions. The sensory evaluation of a cosmetic takes place based on the visual, olfactory and haptic impressions.
Visual impressions: all features that can be perceived visually (color, shape, structure)
Olfactory impressions: all olfactory impressions than can be perceived by drawing air in through the nose, which can frequently be differentiated in the initial scent (headnote), main scent (middle note, body) and afterscent (final note). The volatile substances not released until use also contribute to the olfactory impression.
Haptic impressions: all sensations of the sense of touch, which relate primarily to structure and consistency of the product.

The sensory analysis utilizes the possibility of detecting the overall sensory impression of a product in an integral manner. The disadvantages of the sensory analysis are the subjectivity of the impression, the ability of test persons to be easily influenced and the large spread of results caused thereby. These weaknesses are counteracted nowadays by the use of groups of trained test persons, reciprocal shielding of the examiners and statistical evaluation of the usually numerous analysis data.

The method of sensory analysis most frequently used in research and development is comparison testing. The objective is hereby usually limited to the identification of a pattern deviating from several samples or from a control sample. While with comparison tests only two samples are compared to one another within a test, with the ranking method a sequence of three and more samples is to be established, namely as a rule according to intensity, quality, popularity or the like with a comparison sample. This (simple) method is suitable, e.g. for a preliminary selection of samples in product optimization and is also frequently used in market research.

The constituents of the texture can be described as follows:

Mechanical properties: Reaction to stress, measured kinesthetically

Hardness: Force, in order to achieve a given deformation

| Food | Skin care | Materials |
|---|---|---|
| Strength (compression) | Compressive force | Compressive force |
| Hardness (bite) | Elastic force | Separation force |

Cohesion: Degree until the sample piece is deformed

| Food | Skin care | Materials |
|---|---|---|
| Cohesive | Cohesive | Stiffness |
| Tough | Short | Separation force |
| Break-resistant (crispy) | | |
| Viscosity | Viscosity | |

Adhesion: Force necessary to remove the sample piece from a surface

| Food | Skin care | Materials |
|---|---|---|
| Sticky (teeth/palate) | Sticky | Material-material friction |
| Teeth adhesion | Dragging | Hand friction (dragging) |

Density: Compactness of the cross section

| Food | Skin care | Materials |
|---|---|---|
| Dense/heavy | Dense/heavy | Bulk/delicate |
| Airy/bulky/light | Airy/light | |

Elasticity: Return rate to starting condition after some deformation

| Food | Skin care | Materials |
|---|---|---|
| Elastic/rubbery | Elastic | elastic |

Geometric properties: perception of the particles (size, shape, alignment) by touch means

Smoothness: no particles present

Gritty: small hard particles

Grainy: Small particles

Chalky/powdery: fine particles (film)

Fibrous: long stringy particles (fibrous material)

Clumpy/uneven: large uniform particles or projections

Moisture properties: Perception of water, oil, fat measured by touch means, moisture: quantity of wetness/oiliness if it is not certain whether it is oil and/or water,

moisture release: quantity of the wetness/oiliness released

| Food | Skin care | Materials |
|---|---|---|
| Juicy | Moisture deposit | Moisture release |

Oily: quantity of liquid fat

Fatty: quantity of solid fat

In every case the terminology is specific to each product type, but it is based on the underlying rheological properties according to the first texture profile publications (Szczesniak, 1963: Szczesniak et al., 1963; Brandt et al., 1963).

The test persons are selected on the basis of their ability to distinguish known structural differences in the specific product application for which the test persons are to be trained (solid foods, beverages, semi-solid bodies, skin care products, fabrics, paper etc.). As with most other descriptive analysis techniques, the test persons are interviewed in order to determine interest, availability and attitude.

The test persons who are selected for training are exposed to a large selection of products from the category to be examined, in order to have a broad reference system. In addition, the test persons are introduced to the basic structural principle that is involved in the structure of the products to be tested. This learning experience provides the test persons with an understanding of the terms of mechanical input forces and produced stress of the product.

The test persons can thus avoid lengthy discussions about redundant expressions and select the technically most appropriate and illustrative expressions for evaluating products. The test persons also define all expressions and all procedures for evaluation and thus reduce in part the changeability of most descriptive tests. The reference keys that are used in training the test persons can later act as reference points for approximating scale values with further reduction of the changeability of the test persons.

The test pieces are evaluated by each test person independently using one of the scaling techniques discussed above.

The original texture profiling method uses an expanded 13-point version of the aroma profile scale.

However, in recent years texture profile test persons have been trained using category, linear and ME scales, for food texture references to the use with a 15-point or 15-cm linear scale. Depending on the type of scale used by the test persons and according to the manner in which the data must be treated, the evaluations by the test persons can come from a group consensus, as with the aroma profile method, or from a statistical analysis of the data. For final reports, the data can be shown in table or graphic form.

One object of the present invention was therefore to find preparations which in addition to the usual criteria for cosmetics, such as compatibility, storage stability and the like, also provide hitherto unknown cosmetic benefits important to the consumer. In particular, the sought preparations are to be suitable for a use in the field of personal hygiene, i.e., with an application to the entire body (and thus distinguished from a purely facial skin care application, in a relatively large quantity), but at the same time are thereby attractive in sensory terms for application in the facial area. This manifests itself in particular in good distribution, rapid absorption and in a rich and nurturing impression.

These objects are attained according to the invention by cosmetic or dermatological emulsions with a content of one or more acyl arginates having the formula

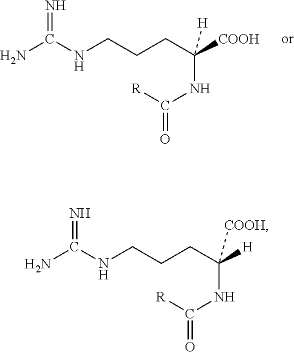

wherein R can have values from 5 through 22 and preferably represents an unbranched alkyl chain of 11 C atoms (=lauroyl arginate).

It is possible, while retaining the features that characterize the present invention, to obtain cosmetically sophisticated emulsions, which are characterized by extremely pleasant sensory characteristics and cosmetic impression.

The contact wetting angle measurement (described in Cosmetics & Toiletries, 01/2007, Vol. 122 No. 1, pages 20-27 “Correlating Water Contact Angles and Moisturization/Sensory Claims”) is suitable as a measuring method for characterizing various formulations regarding the light feeling on the skin, easy distribution and non-oily feeling.

Surprisingly, it has now been shown that through the use of acyl arginates such as, for example, lauroyl arginate (LAE, ethyl lauroyl arginate HCl, CAS 60372-77-2), the contact wetting angle of a sunscreen preparation is smaller than without. It can be concluded from this that the sensory properties of the formulation are better than without lauroyl arginate.

Advantageously, the preparations according to the invention can be water-in-oil emulsions, particularly preferably oil-in-water emulsions.

Advantageously, preparations according to the invention contain the acyl arginates from 0.001 to 10% by weight, preferably 0.05 to 5% by weight, in particularly preferably 0.05 to 1% by weight, in each case based on the total weight of the preparations.

The preparations according to the invention are extremely satisfactory preparations in every respect. It was not foreseeable to one skilled in the art that emulsions which through the targeted adjustment of the pseudoplastic flow behavior (structural viscosity) and thixotropy would exhibit the properties according to the invention despite relatively high yield point, in particular can be distributed on the skin very easily, are absorbed quickly, yet are very rich and conditioning.

The cosmetic preparations according to the invention can be composed as usual and used to treat and/or care for the skin and as a cosmetic product in decorative cosmetics.

The cosmetic and/or dermatological formulations according to the invention can be composed as usual and can be used to treat the skin and/or hair in terms of a dermatological treatment or a treatment in terms of conditioning cosmetics. However, they can also be used in cosmetic products in decorative cosmetics.

For application, the cosmetic and/or dermatological formulations according to the invention are applied to the skin and/or the hair in sufficient quantities in the usual manner for cosmetics and dermatological products.

A content of antioxidants is generally advantageous. The quantity of antioxidants (one or more compounds) in the preparations is then preferably 0.001 to 30% by weight, particularly preferably 0.05-20% by weight, in particular 1-10% by weight based on the total weight of the preparation.

The emulsions used for the purpose of the invention can be based on one or more of the following emulsifiers.

Glyceryl stearate citrate, glyceryl stearate (self-emulsifying), stearic acid, stearate salts, polyglyceryl-3 methyl glucose distearate, ceteareth-20, PEG-40 stearate, PEG 100 stearate, sucrose polystearate in combination with hydrogenated polyisobutene, sodium stearoyl glutamate, cetearyl alcohol in combination with PEG-40 hydrogenated castor oil, sodium cetearyl sulfate, glyceryl stearate, potassium cetyl phosphate, polyglyceryl-3 sorbitan stearate, silicone polyether, copolymers such as, for example, PEG-10 dimethicone, PEG/PPG-14/4 dimethicone, glyceryl stearate citrate and/or sodium stearoyl glutamate, ceteareth-n where n=5-50, sorbitan oleate, distearyl dimonium chloride, stearyl dimethicone, lecithin, sodium dihydroxycetyl phosphate, PG-10 stearate, lauryl PEG/PPG-18/18 methicone, cetyl PEG/PPG-10/1 dimethicone, polyglyceryl-2 caprate, isostearyl diglyceryl succinate, cetyl phosphate, sorbitan sesquioleate, polyglyceryl-3 methylglucose sesquistearate, polyglyceryl-3 methylglucose distearate, potassium cetyl phosphate, polyglyceryl-2 sesquiisostearate, glyceryl isostearate, behentrimonium chloride, cetearyl glucoside, PEG/PPG-18/18 dimethicone, alkoxylated silicone emulsifiers in general, polyglycerin derivatized silicone emulsifiers in general, polycaprolactone, sucrose polycottonseedate, acetyl trifluoromethylphenyl valylglycine, hydroxyethyl piperazine, ethanesulfonic acid, hydroxypropyl tetrahydropyrantriol, tocopheryl glucoside, guanosine, polyester-5, tri C14-15 alkylcitrate, PEG/PPG-14/7 dimethyl ether, sodium dodecylbenzene sulfonate, chrysanthenum parthenium, sodium hydroxypropyl starch phosphate, PEG-60 glyceryl isostearate, succinoglycan, polyglyceryl-2 caprate, dodecene, oxothiazolidinecarboxylic acid, eucalyptol, hydrogenated myristyl olive esters, hydrolyzed wheat protein PG-propyl methylsilanediol.

The lipid phase can be advantageously selected from the following group of substances:
Mineral oils and mineral waxes;
Oils, such as triglycerides in general and specifically of capric or of caprylic acid, furthermore natural oils such as e.g., castor oil;
Fats, waxes and other naturally occurring and synthetic fatty substances, preferably esters of fatty acids with alcohols of low C number, for example with isopropanol, propylene glycol or glycerol, or esters of fatty alcohols with alkanoic acids of low C number or with fatty acids;
Alkyl and/or benzoates;
Silicone oils, such as dimethylpolysiloxanes, diethylpolysiloxanes, diphenylpolysiloxanes, disiloxane dimethicones and mixed forms thereof.

The oil phase of the emulsions in the context of the present invention is advantageously chosen from the group of esters of saturated and/or unsaturated, branched and/or unbranched alkane carboxylic acids having a chain length of 3 to 30 C atoms and saturated and/or unsaturated, branched and/or unbranched alcohols having a chain length of 3 to 30 C atoms, from the group of esters of aromatic carboxylic acids and saturated and/or unsaturated, branched and/or unbranched alcohols having a chain length of 3 to 30 C atoms. Such ester oils can then advantageously be chosen from the group of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, octyl palmitate, octylcocoate, octyl isostearate, octyl dodecyl myristate, isopropyl lauroyl sarcosinate, dibutyl adipate, cetearyl isononanoate, isopropyl myristate, stearyl heptanoate as well as synthetic, semi-synthetic and naturally occurring mixtures of such esters, for example jojoba oil. Further oils which can be used advantageously for the purpose of the present invention are: dialkylethers and dialkyl carbonates in particular dicaprylyl carbonate, neopentyl glycol diheptanoate, caprylic/capric/diglycerylsuccinate, butylene glycol dicaprylate/dicaprate, C12-13-alkyl lactate, di-C12-13-alkyltartrate, triisostearin, dipentaerythrityl hexacaprylate/hexacaprate, propylene glycol monoisostearate, tricaprylin, dimethylisosorbid, caprylic/capric octyldodecanol, isopropyl palmitate, ethylhexyl cocoate, butyrospermum parkii, hydrogenated coco-glycerides, simmondisia chinensis oil, tridecyl stearate, tridecyl trimellitate, dipentaerythrityl hexacaprylate/hexacaprate, lanolin alcohol, echium lycopsis oil, shorea robusta butter.

The aqueous phase of the preparations according to the invention, if present, advantageously likewise comprises alcohols, diols and/or polyols having a low C number, and ethers thereof, preferably ethanol, isopropanol, propylene glycol, glycerin, ethylene glycol, ethylene glycol monoethyl- or -monobutylether, propylene glycol monomethyl, monoethyl or monobutylether, diethylene glycol monomethyl or monoethyl ether and analogous products, also alcohols having a lower C number, e.g., ethanol, isopropanol, 1,2-propane diol.

Very particularly advantageously, the preparation according to the invention contains moisturizers, such as glycerin, namely in particular in concentrations of approx. 2-50% by weight, wherein the preferred embodiments are characterized by glycerin contents of 25-35, very particularly preferably approx. 30% by weight. However, it is also possible to achieve glycerin contents of approx. 70% by weight without cosmetic or physical disadvantages having to be tolerated.

Further moisturizers for the purpose of the invention are urea and derivatives of urea (such as Hydrovance®) lactic acid, hyaluronic acid, AHAs, EHAs, sodium salt of pyroglutamic acid, xylitol, serine, sodium acetate, ectoin, chitosan and corresponding derivatives of the cited compounds, collagen, plankton, extract of imperata cylindra (Moist 24®) acrylic acid homopolymers (Lipidure-HM®) beta glucan and corresponding derivatives, natural oils such as apricot oil. All of the referenced substances can optionally also be used in encapsulated form.

As a propellant for preparations that can be sprayed from aerosol containers, the usual known volatile liquefied propellants, for example hydrocarbons (propane, butane, isobutane) are suitable, which can be used alone or mixed with one another. Compressed air can also be used advantageously.

Advantageously, preparations according to the invention can comprise other substances which absorb UV radiation in the UVB and/or UVA range, the total amount of the filter substances being, for example, 0.1% to about 30% by weight, preferably 0.5% to 10% by weight, in particular 1.0% to 6.0% by weight, based on the total weight of the preparations, in order to provide cosmetic preparations that protect the hair or the skin from the entire range of ultraviolet radiation. They can also serve as sunscreen agents for the hair or the skin.

The cosmetic preparations according to the invention can furthermore advantageously, although not necessarily, comprise fillers or siloxane elastomers which, e.g., further improve the sensory and cosmetic properties of the formulations and, for example, produce or intensify a velvety or silky feeling on the skin.

Advantageous fillers in the context of the present invention are starch and starch derivatives, pigments which have neither a chiefly UV filter nor a coloring action (such as for example boron nitride etc.) and Aerosils(R) (CAS No. 7631-86-9) and/or talcum.

The following can be employed particularly advantageously for the purpose of the present invention: tapioca starch, distarch phosphate, aluminum- or sodium-starch octenyl succinate and the like, polymethylsilesquioxane, emulsifying silicone elastomers, non-emulsifying silicone elastomers, polymethylmethacrylates, polymethylmethacrylate crosspolymer, polyamide, nylon, polyethylene powder, polydimethylsiloxyethyl dimethicone, acrylates/C10-30 alkyl acrylate crosspolymer, polysaccharide or derivatives thereof, e.g., cellulose and cellulose derivatives, e.g., hydroxypropyl methyl cellulose, methyl cellulose, carboxymethyl cellulose, hydroxyethyl cellulose, hyaluronic acid, xanthan gum, polymers from the group of polyacrylates, preferably a polyacrylate from the group of the so-called carbopols, for example carbopols type 980, 981, 1382, 2984, 5984, respectively individually or in combination. Further advantageous thickeners according to the invention are those with the INCI name acrylates/C10-30 alkyl acrylate crosspolymer (e.g. Pemulen TR1, Pemulen TR2, Carbopol 1328 from NOVEON) as well as Aristoflex AVC (INCI: ammonium acryloyldimethyltaurate/VP copolymer) as well as a copolymer of vinylpyrrolidone and acrylic acid, PEG-10 dimethicone/vinyl dimethicone crosspolymer, nylon 66, poly C10-30 alkyl acrylate, gums, polysaccharides, cellulose derivatives, phyllosilicates, polyacrylates and/or other polymers, polyacrylamides (Seppigel 305), polyvinyl alcohols, PVP, PVP/VA copolymers, polyglycols, gum arabic, carob flour, tragacanth, karaya, guar gum, pectin, gellan gum, carrageenan, agar, algin, chondrus, xanthan gum, derivatized gums, such as hydroxypropyl guar (Jaguar® HP 8), chitin and chitosan, chondroitin sulfate, starch and starch derivatives, montmorillonite, bentonite, hectorite, laponite, magnesium aluminum silicates such as Veegum®. These can be used per se or in modified form, such as stearylalkonium hectorite. Furthermore, silica gels can also be used advantageously.

The following examples are intended to clarify the invention without restricting it.

| | Example | |
|---|---|---|
| | 1 | 2 |
| Glyceryl stearate citrate | 2.5 | 3 |
| Stearyl alcohol | 2 | |
| Carbomer | | 0.3 |
| Xanthan gum | 0.4 | 0.2 |
| C18-38 Acid triglyceride | 0.5 | 1 |
| Myristyl myristate | 1 | 2 |
| Butylene glycol dicaprylate/dicaprate | | 3 |
| Propylheptyl caprylate | 5 | 5 |
| Octyldodecanol | | 2 |
| Cyclomethicone | 2 | 3 |
| Butylene glycol | 5 | |
| Propylene glycol | | 1 |
| Glycerin | 3 | 3 |
| Octane-1,2-diol | 2 | 5 |
| Bis-ethylhexyloxyphenol methoxyphenyl triazine | 2 | 3 |
| Tris-(biphenyl)-1,3,5-triazine. | 2 | |
| Butyl methoxydibenzoylmethane | 3 | 2 |
| Octocrylene | 5 | 5 |
| Ethylhexyl triazine | | 3 |
| Ethylhexyl methoxycinnamate | 0.5 | |
| Titanium dioxide | 2 | |
| PVP/hexadene copolymer | 0.2 | 0.1 |
| Vitamin E acetate | 0.2 | 0.2 |
| Na₂H₂EDTA | 0.1 | 0.2 |
| Perfume | 0.2 | 0.3 |
| Lauroyl arginate | 0.25 | 0.1 |
| Phenoxy ethanol | 0.7 | 1 |
| Sodium hydroxide | q.s. | q.s |
| Water | Ad 100 | Ad 100 |
| | Example | |
| | 3 | 4 |
| Glyceryl stearate SE | 1 | 1 |
| Cetearyl alcohol + PEG-40 castor oil + sodium | 2.5 | 2.5 |
| cetearyl sulfate | | |
| Stearyl alcohol | | 1 |
| Cetyl alcohol | | 3 |
| Acrylates/C₁₀₋₃₀ alkyl acrylate cross polymer | 0.1 | 0.1 |
| Xanthan gum | 0.2 | 0.3 |
| C18-38 Acid triglyceride | | 1 |
| C₁₂₋₁₅ Alkyl benzoate | 4 | 5 |
| Myristyl myristate | | 1 |
| Butylene glycol dicaprylate/dicaprate | 7 | 3 |
| Propylheptyl caprylate | | 12 |
| Dicaprylyl ether | 5 | 2 |
| Cyclomethicone | 1 | |
| Dimethicone | 8 | |
| Emulsifying elastomer | 1 | |
| Butylene glycol | 3 | |
| Propylene glycol | | 5 |
| Glycerin | | 3 |
| Octane-1,2-diol | 2 | |
| 2-Methyl-1,3 propane diol | 3 | 1 |
| Bis-ethylhexyloxyphenol methoxyphenyl triazine | 0.5 | 4 |
| Butyl Methoxydibenzoylmethane | | 4 |
| Homosalate | 4 | |
| Octyl salicylate | 3 | |
| Octocrylene | 8 | 8 |
| Ethylhexyl methoxycinnamate | 0.5 | |
| Dioctyl butamido triazine | | 2 |
| Titanium dioxide | | 2 |
| PVP/hexadecane copolymer | 0.2 | |
| Vitamin E acetate | 0.3 | 0.1 |
| Na₂H₂EDTA | 0.2 | 0.2 |
| Perfume | 0.4 | 0.25 |
| Lauroyl arginate | 0.4 | 0.5 |
| Benzethonium chloride | 0.05 | |
| Dehydroacetic acid | | 0.5 |
| Ethylhexyl glycerin | 0.5 | |
| Sodium hydroxide | q.s. | q.s. |
| Water | Ad 100.0 | Ad 100.0 |
| | Example | |
| | 5 | 6 |
| Sodium stearyol glutamate | 0.1 | |
| Sucrose polystearate | 0.8 | |
| Copolymer of vinylpyrrolidone and acrylic acid | 0.3 | 0.8 |
| Acrylate/C10-30 alkyl acrylate crosspolymer | 0.2 | |
| Butylene glycol dicaprylate/dicaprate | | 3 |
| Octyldodecanol | | 3 |
| Dicaprylyl carbonate | 3 | |
| Isodecyl neopentanoate | 2 | |
| Cyclomethicone | | 8 |
| Dimethicone crosspolymer | | 1 |
| Glycerin | 5 | 5 |
| Hexane-1,2 diol | 2 | 3 |
| Bis-ethylhexyloxyphenol methoxyphenyl triazine | 1 | |
| Butyl methoxydibenzoylmethane | 3 | 2 |
| Diethylamino hydroxybenzoyl hexyl benzoate | | 2 |
| Polysilicone 15 | 2 | |
| Octyl salicylate | | 5 |
| Octocrylene | 4 | 8 |
| Phenylbenzimidazole sulfonic acid | 2 | |
| Ethanol | | 4 |
| Lauroyl arginate | 0.3 | 0.1 |
| DMDM hydantoin | 1 | |
| 1,2-Hexanediol | | 0.8 |
| Pentylene glycol | | 3 |
| Water | Ad 100.0 | Ad 100.0 |
| | Example | |
| | 7 | 8 |
| Sodium stearoyl glutamate | 0.4 | 0.1 |
| Sucrose polystearate | 0.6 | 0.5 |
| Cetearyl alcohol | | 1 |
| Copolymer of vinyl pyrrolidone and acrylic acid | 0.7 | |
| Acrylate/C10-30 alkyl acrylate crosspolymer | | 0.2 |
| Carbomer | | 0.2 |
| C12-15 Alkyl benzoate | | 2 |
| Caprylic/capric triglyceride | | 2 |
| Cyclomethicone | 3 | 24 |
| Dimethicone | 2 | |
| Dimethicone crosspolymer | | 3 |
| Glycerin | 7 | 1 |
| Polymethylsilsesquioxane | 5 | |
| Bis-ethylhexyloxyphenol methoxyphenyl triazine | 2 | 2.5 |
| Ethylhexyl triazine | | 1 |
| Diethylamino hydroxybenzoyl hexyl benzoate | 5 | 3 |
| Ethylhexyl methoxycinnamate | 5 | |
| Octylsalicylate | | 3 |
| Homosalate | | 5 |
| Octocrylene | 2 | 5 |
| Ethanol | | 3 |
| Lauroyl arginate | 0.25 | 0.4 |
| Methylisothiazolinone | 0.05 | |
| DMDM hydantoin | | 0.5 |
| Water | Ad 100.0 | Ad 100.0 |
| Hydrodispersion | 9 | 10 |
| Sodium carbomer | 0.30 | |
| Copolymer of vinylpyrrolidone and acrylic acid | | 1.0 |
| Sodium stearoyl glutamate | | 0.1 |
| Acrylates/C10-30 alkyl acrylate crosspolymer | 0.10 | |
| Xanthan gum | | 0.50 |
| 2-(4-Diethylamino-2′-hydroxybenzoyl) benzoic | 2.00 | 0.25 |
| acid hexyl ester | | |
| Terephthalylidene dicamphor sulfonic acid | | 2.0 |
| Ethylhexyl triazine | | 4.00 |
| Butyl methoxydibenzoyl methane | | 4.2 |
| Titanium dioxide | | 0.50 |
| 1,2-Hexane diol | | 0.50 |
| Methylpropane diol | 3 | 0.5 |
| C₁₂₋₁₅ Alkyl benzoate | | 2.00 |
| Oleic acid | | 0.5 |
| Butyleneglycol dicaprylate/dicaprate | 6.00 | 4.00 |
| PVP hexadecene copolymer | 0.50 | 0.50 |
| Glycerin | 5.00 | 10.00 |
| 1,2-Octane diol | 0.1 | |
| Vitamin E acetate | 0.5 | 0.50 |
| Panthenol | 0.25 | 1.50 |
| Trisodium EDTA | 0.20 | 20 |
| Ethanol | | 3.00 |
| Lauroyl arginate | 0.5 | 0.2 |
| Methylisothiazolinone | | 0.09 |
| DMDM hydantoin | 0.5 | |
| Perfume, dyes | q.s. | q.s. |
| Water | Ad 100 | Ad 100 |

1.-5. (canceled)

6. A cosmetic or dermatological emulsion, wherein the emulsion comprises one or more acyl arginates of one or more of the following formulae: 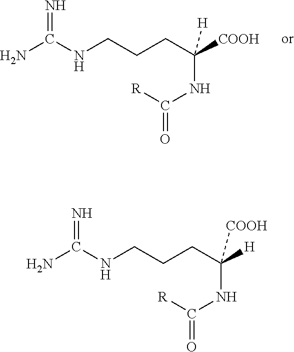
wherein R comprises an unbranched alkyl chain having from 5 to 17 carbon atoms.

7. The emulsion of claim 6, wherein the emulsion comprises lauroyl arginate.

8. The emulsion of claim 6, wherein the emulsion comprises from 0.001% to 10% by weight of the one or more acyl arginates, based on a total weight of the emulsion.

9. The emulsion of claim 8, wherein the emulsion comprises from 0.05% to 5% by weight of the one or more acyl arginates.

10. The emulsion of claim 8, wherein the emulsion comprises from 0.05% to 1% by weight of the one or more acyl arginates.

11. The emulsion of claim 6, wherein the emulsion is an oil-in-water emulsion.

12. The emulsion of claim 6, wherein the emulsion is a water-in-oil emulsion.

13. The emulsion of claim 6, wherein the emulsion further comprises glycerin.

14. The emulsion of claim 13, wherein the emulsion comprises from 2% to 50% by weight of glycerin.

15. The emulsion of claim 13, wherein the emulsion comprises from 25% to 35% by weight of glycerin.

16. The emulsion of claim 13, wherein the emulsion comprises about 30% by weight of glycerin.

17. A cosmetic or dermatological emulsion, wherein the emulsion comprises lauroyl arginate.

18. The emulsion of claim 17, wherein the emulsion is an oil-in-water emulsion.

19. The emulsion of claim 18, wherein the emulsion further comprises glycerin.

20. The emulsion of claim 19, wherein the emulsion comprises from 2% to 50% by weight of glycerin.

21. The emulsion of claim 19, wherein the emulsion comprises from 25% to 35% by weight of glycerin.

22. The emulsion of claim 19, wherein the emulsion comprises about 30% by weight of glycerin.

23. The emulsion of claim 17, wherein the emulsion further comprises glycerin.

24. The emulsion of claim 23, wherein the emulsion comprises from 25% to 35% by weight of glycerin.

25. The emulsion of claim 23, wherein the emulsion comprises about 30% by weight of glycerin.
